(19) United States
(12) Patent Application Publication
(10) Pub. No.: US 2021/0205452 A1
(43) Pub. Date: Jul. 8, 2021

(54) Composition of Concentrated Human Immunoglobulins

(71) Applicant: Laboratoire Francais du Fractionnement et des Biotechnologies, Les Ulis (FR)

(72) Inventor: Cecile JAUME, Soisy sur Seine (FR)

(73) Assignee: Laboratoire Francais du Fractionnement et des Biotechnologies, Les Ulis (FR)

(21) Appl. No.: 17/056,626

(22) PCT Filed: May 23, 2019
(86) PCT No.: PCT/FR2019/051195

§ 371 Date: Nov. 18, 2020

(30) Foreign Application Priority Data
May 24, 2018 (FR) 1854412

(51) Int. Cl. A61K 39/395; C07K 16/00

(52) U.S. Cl. CPC A61K 39/39591; C07K 16/00

(57) ABSTRACT

The invention proposes the use of a pharmaceutical composition comprising 200 g/L immunoglobulin G (IgG), between 200 and 250 mM glycine and between 15 and 25 ppm nonionic detergent particularly suitable for subcutaneous administration. In addition, the pH of the composition is between 4.6 and 5.0.

The invention relates to a concentrated human immunoglobulin G composition having improved stability over time. The composition according to the invention is particularly suitable for subcutaneous use.

A number of pathologies are currently treated with immunoglobulin G (IgG) compositions. For example, mention may be made of primary immune deficiencies with deficient antibody production, Kawasaki disease, immune thrombocytopenic purpura in children and adults, secondary immune deficiencies with deficient antibody production, in particular chronic lymphocytic leukemia or myeloma associated with repeated infections, HIV infection in children associated with bacterial infections, multifocal motor neuropathies, Guillain-Barré syndrome, acute severe or chronic Parvovirus B19 infections, acquired or constitutional immunodeficiency, corticosteroid-resistant dermatomyositis, acute myasthenia gravis, chronic idiopathic polyradiculoneuritis, immune thrombocytopenic purpura, for example associated with HIV infection, stiff-man syndrome, autoimmune neutropenia, resistant autoimmune erythroblastopenia, autoantibody-acquired anticoagulation syndrome, rheumatoid arthritis, uveitis, etc.

For the treatment of certain pathologies, the use of IgG compositions suitable for subcutaneous administration (SCIg) may prove particularly advantageous. This route of administration offers patients greater flexibility and independence, improving their quality of life. To this end, immunoglobulin compositions with high IgG concentrations have been developed. However, it is known that as IgG concentration increases, problems of stability over time increase. In particular, a higher formation of oligomers and polymers is observed in such compositions. Oligomers and polymers are likely to activate the complement system with associated risks of anaphylactic reactions. These oligomers and polymers are also likely to induce hypotension phenomena in treated patients. This is undesirable and is strictly controlled from a regulatory point of view. In addition, the formation of protein aggregates, due in particular to thermal, mechanical or chemical stress, contributes to these instability problems. In addition, it is known that the presence of certain detergents conventionally used for intravenous administration can induce a local reaction when administered subcutaneously.

To date, concentrated IgG compositions, i.e. comprising at least 16% IgG, for subcutaneous administration are not fully satisfactory, in particular, in terms of stability and local tolerance.

In this context, there is still a need for easy-to-use high-concentration IgG compositions.

### SUMMARY OF THE INVENTION

By working on the stability and tolerance issues specific to concentrated IgG compositions for subcutaneous administration, the Applicant demonstrated that it is possible to obtain concentrated IgG compositions which have very good local tolerance after subcutaneous administration and are advantageously particularly stable over time. More precisely, the Applicant developed a formulation combining a high concentration of IgG with glycine and a nonionic detergent, wherein the glycine and the nonionic detergent are present at particularly low concentrations, suitable for subcutaneous use. The combination of nonionic detergent and glycine in such proportions contributes to a high stability of said formulation over time. In addition, the Applicant demonstrated that such a formulation is particularly well tolerated by patients when administered subcutaneously. Advantageously, the concentrated immunoglobulin compositions according to the invention have a substantially physiological osmolality. Advantageously, no excipients other than glycine and nonionic detergent are required, and in particular no acetate, mannitol or albumin, to guarantee greater stability during storage.

An object of the invention is therefore a pharmaceutical composition comprising:
200 g/L±5% immunoglobulin G (IgG)
between 200 and 250 mM glycine
between 15 and 25 ppm nonionic detergent
   the pH of the composition being between 4.6 and 5.0.

In a particular embodiment, the composition consists only of water, IgG, glycine and nonionic detergent.

The composition according to the invention advantageously comprises 20% (200 g/L) IgG. Generally, in the context of the invention, the IgG concentrations are ±5% of the concentration in g/L.

According to a preferred embodiment, the IgG are human IgG, in particular obtained from plasma or from plasma fractions.

In a particular embodiment, the glycine concentration is 215 mM, ±5%. Such a glycine concentration is particularly suitable for 20% IgG compositions for subcutaneous use.

In a particular embodiment, the nonionic detergent is selected from poloxamers, and in particular Pluronic F68®, and polysorbates, preferably polysorbate 20 or polysorbate 80, even more preferably polysorbate 80.

Preferentially the nonionic detergent is at a concentration of about 20 ppm±10%.

The invention also has as object such an IgG composition for use subcutaneously for the treatment of immune system dysfunction, autoimmune and/or inflammatory disease, infection or neurological disease.

### DETAILED DESCRIPTION

### Definitions

In the context of the invention, “immunoglobulin G” or “IgG” means polyvalent immunoglobulins that are essentially IgG, possibly including IgM. These may be whole immunoglobulins, or fragments such as F(ab′)2 or F(ab) and any intermediate fraction obtained during the polyvalent immunoglobulin manufacturing process.

The term “stability” corresponds to the physical and/or chemical stability of IgG. The term “physical stability” refers to the reduction or absence of formation of insoluble or soluble aggregate of the dimeric, oligomeric, or polymeric forms of Ig, as well as the reduction or absence of any structural denaturation of the molecule. The term “chemical stability” refers to the reduction or absence of any chemical modification of IgG during storage, in solid or dissolved form, under accelerated conditions. For example, hydrolysis, deamidation, and/or oxidation phenomena are avoided or delayed. Oxidation of sulfur-containing amino acids is limited. The stability of an IgG composition can be assessed by visual inspection using in particular a fiber optic device (opalescence, particle formation), by measuring turbidity using a spectrophotometer measuring absorbance or optical density at 400 nm, for example, and/or by measuring dynamic light scattering (DLS), which allows the measurement of particles in solution with sizes between about 1 nm and 1 μm.

In the context of the invention, the expression “between x and y” means that the x and y values are included.

### Formulations:

Preferably, the IgG concentration is 200 g/L, ±5%.

In the context of the present invention, the concentrations refers to the concentrations in the final, ready-to-use, composition. The concentrations are determined with respect to compositions in liquid form, before desiccation, or after reconstitution in the form of an injectable preparation.

Particularly advantageously, the Applicant demonstrated that it is possible to obtain compositions comprising 20% IgG that are particularly stable over time by using a minimum of excipients. Thus, according to the invention, the 20% IgG compositions advantageously comprise between 200 and 250 mM glycine, preferably between 200 and 230 mM, preferentially between 210 and 220 mM. In a particular embodiment, the 20% IgG composition comprises 215 mM glycine, ±5%.

Similarly, the 20% IgG compositions comprise between 15 and 25 ppm nonionic detergent. In a particular embodiment, the 20% IgG composition comprises 20 ppm nonionic detergent, ±10%.

In an embodiment, the nonionic detergent used in the composition according to the invention is advantageously selected from polysorbates and in particular among polysorbate 80 (or Tween®80, which is polyoxyethylene sorbitan monooleate), and polysorbate 20 (or Tween®20, which is polyoxyethylene sorbitan monolaurate). In another embodiment, the nonionic detergent is selected from poloxamers, in particular Pluronic®F68 (polyethylene-polypropylene glycol). In another embodiment, the nonionic detergent is selected from Triton® X 100 (octoxinol 10), polyoxyethylene alkyl ethers and ethylene/polypropylene block copolymers. The nonionic detergents can also be combined with each other.

In an embodiment, the composition is free of mannitol and/or albumin and/or acetate. Indeed, the Applicant showed that mannitol and/or acetate and/or albumin are not necessary for the stabilization of a 20% IgG composition.

According to a preferred embodiment, the composition contains neither mannitol nor albumin. According to another preferred embodiment, the composition does not contain acetate. According to a particularly preferred embodiment, the composition according to the invention contains neither mannitol, acetate, nor albumin. In an alternative or complementary embodiment, the composition does not contain sugar.

Advantageously, the Applicant has demonstrated that the compositions according to the invention have an osmolality particularly adapted to administration by injection, in particular subcutaneous, and this with no additional number and/or amount of excipients. Thus, the invention proposes 20% IgG compositions having a measured osmolality between about 300 and 400 mOsm/kg, adjusted with glycine. In the context of the invention, and unless otherwise stated, the osmolality of the composition means the osmolality measured in said composition.

Osmolality is advantageously measured using an osmometer calibrated with standard solutions, in particular according to the method recommended by the European Pharmacopoeia, (European Pharmacopoeia 5.0 of 2005-01/2005:2.2.35.). Of course, any other method of measuring osmolality can be used.

In a preferred embodiment, the only excipients of the 20% IgG composition according to the invention are glycine and nonionic detergent. Such a formulation allows a good stabilization of immunoglobulin compositions over time and a reduction of preparation times and costs on an industrial scale thanks to the presence of a minimum effective number and amount of excipients. Advantageously, such a composition has an osmolality compatible with administration by injection, in particular subcutaneously.

In a particular embodiment, the composition consists essentially of IgG, glycine, a nonionic detergent and water, in the sense that any other excipient that may be present would be present in trace amounts only.

According to the invention, the final pH of the composition is advantageously between 4.6 and 5.0. Preferentially, the pH is about 4.8±0.1. A pH of 4.8±0.1 gives particularly satisfactory results in terms of stability over time. The final pH refers to the pH of the composition after formulation, i.e. in the ready-to-use composition. Unless otherwise stated, in the present description, the pH of the composition refers to the final pH.

A preferred IgG composition according to the invention comprises:
200 g/L, ±5%, IgG
200 to 250 mM glycine,
15 to 25 ppm nonionic detergent, the pH of the composition being between 4.6 and 5.0.

Another preferred IgG composition according to the invention comprises:
200 g/L, ±5% IgG
200 to 230 mM glycine,
15 to 25 ppm nonionic detergent, preferentially polysorbate 80 or Pluronic F68®
   the pH of the composition being between 4.6 and 5.0.

A particularly preferred IgG composition according to the invention comprises:
200 g/L, ±5% IgG
215 mM glycine, ±5%.
20 ppm nonionic detergent, ±10%, preferentially polysorbate 80 or Pluronic F68®,
   the pH of the composition being 4.8±0.1.

Another particularly preferred IgG composition according to the invention comprises:
200 g/L IgG
about 215 mM glycine
about 20 ppm nonionic detergent, preferentially polysorbate 80 or Pluronic F68®
   the pH of the composition being 4.8.

The measured osmolality of this 20% IgG composition is advantageously about 300-400 mOsm/kg, ±2%, preferably about 340 mOsm/kg, ±2%.

In a particularly advantageous embodiment, the composition according to the invention comprises
200 g/L IgG
about 215 mM glycine
about 20 ppm nonionic detergent, preferentially polysorbate 80 or Pluronic F68®
   the pH of the composition being 4.8, and its osmolality being 340 mOsm/kg, ±2%, said composition being devoid of acetate salts, mannitol and albumin.

Surprisingly and advantageously, the Applicant demonstrated that a glycine concentration of about 215 mM, ±5% combined with a polysorbate 80 or Pluronic F68® concentration of 20 ppm, is sufficient to maintain the stability of the 20% immunoglobulin composition over time, while maintaining an osmolality of between 300 and 400 mOsm/kg in the composition, whereas higher concentrations would have been expected to guarantee stability, increasing in parallel the osmolality of said compositions. However, excessive osmolality can cause dehydration of the cells (outflow of intracellular water to the extracellular medium) which is detrimental to the patient. Furthermore, an increase in the amount of excipients, in particular nonionic detergents, can lead to a decrease in the local tolerance of the composition administered subcutaneously. The composition developed by the Applicant, in which the number and amount of excipients are low, is therefore particularly advantageous for subcutaneous administration.

The compositions according to the invention advantageously comprise human immunoglobulin G. Human IgG are generally obtained by fractionation of human blood plasma and provided in an aqueous medium. The aqueous medium consists of water for injection (WFI) which may contain pharmaceutically acceptable and IgG-compatible excipients. IgG compositions may be pretreated with specific virus inactivation/elimination steps, such as solvent detergent treatment, pasteurization and/or nanofiltration. The composition according to the invention comprises IgG which may be polyclonal or monoclonal. IgG can be isolated from human or animal blood or produced by other means, for example by molecular biology techniques, for example in cell systems well known to the person skilled in the art. The composition according to the invention is particularly suitable for highly purified IgG. Advantageously, the IgG of the present invention are obtained by fractionation of human plasma. Preferred human plasma fractionation methods are described by Cohn et al. (J. Am. Chem. Soc., 68, 459, 1946), Kistler et al. (Vox Sang., 7, 1962, 414-424), or Steinbuch et al. (Rev. Franc. Et. Clin. et Biol., XIV, 1054, 1969). A method for preparing an immunoglobulin G composition is also described in the patent application WO2002/092632.

The compositions according to the invention are advantageously in liquid form.

The 20% IgG composition according to the invention, in liquid form and after storage for a period of 6 months at 25° C., has a polymer content much lower than the maximum authorized level set by the European Pharmacopoeia (3%), advantageously less than about 1%.

The compositions of the invention are pharmaceutical compositions, i.e. suitable for therapeutic use. The pharmaceutical compositions of the invention are thus useful as medicinal products, in particular for the treatment of a dysfunction of the immune system, an autoimmune and/or inflammatory disease, an infection or a neurological disease. The compositions according to the invention are particularly suitable for the treatment of disorders such as primary immune deficiencies with deficient antibody production, Kawasaki disease, immune thrombocytopenic purpura in children and adults, secondary immune deficiencies with deficient antibody production, in particular chronic lymphoid leukemia or myeloma associated with repeated infections, HIV infection in children associated with bacterial infections, and multifocal motor neuropathies, Guillain-Barré syndrome, acute severe or chronic Parvovirus B19 infections, acquired or constitutional immunodeficiency, corticosteroid-resistant dermatomyositis, acute myasthenia gravis, chronic idiopathic polyradiculoneuritis, immune thrombocytopenic purpura, for example associated with HIV infection, stiff-man syndrome, autoimmune neutropenia, resistant autoimmune erythroblastopenia, autoantibody-acquired anticoagulation syndrome, rheumatoid arthritis, uveitis. Such use is advantageously by subcutaneous injection.

The composition according to the invention is suitable for the treatment of a human subject, of any age, and more particularly an adult, child or infant subject.

The compositions according to the invention can be advantageously subjected to a method of removing or inactivating infectious agents, for example by solvent-detergent treatment or nanofiltration. Such methods for removing or inactivating infectious agents are well known to the person skilled in the art.

### Routes of Administration:

The 20% IgG composition according to the invention is useful in therapy, and especially in injectable form, in particular subcutaneously.

The subcutaneous route for the treatment of chronic autoimmune diseases has several advantages, such as improved patient comfort and decreased side effects.

Subcutaneous administration does not require venous access, which is, in certain cases, a decisive advantage when the absence of venous access blocks access to treatment, particularly for young children.

The use of subcutaneous immunoglobulins also reduces some of the side effects associated with intravenous infusions, particularly the risk of systemic reactions. The large variations in circulating levels observed with intravenous infusions are avoided, allowing better regulation of serum levels within the physiological range between infusions. Despite a naturally lower bioavailability by the subcutaneous route, subcutaneously administered immunoglobulins (SCIg) have an efficacy at least equivalent to that of intravenously administered immunoglobulins (IVIg).

Finally, the availability of SCIg for home treatment is an important if not decisive advantage for certain treatments. It offers more flexibility and independence to the patient, improving patient quality of life.

Increased concentration contributes to patient comfort by reducing the frequency of injection. The concentration of SCIg is a determining feature that conditions the injection volume and the number of injection sites and consequently the frequency of administration.

The following examples illustrate the invention without limiting its scope.

### EXAMPLES

### Example 1: Accelerated Stress Study of Immunoglobulin Compositions

An immunoglobulin G composition is prepared according to the process as described in the application EP1385886. The product obtained is then concentrated to 200 g/L by ultrafiltration on Ultracel C cellulose membrane (Millipore®) with a 30 kDa cut-off in order to obtain the ready-to-formulate product (RFP).

Glycine, polysorbate 80 or Pluronic F68 is added to the ready-to-formulate 200 g/L concentrates which are adjusted to the desired pH to obtain the following compositions:

TABLE 1
IgG compositions

| | Glycine | Nonionic detergent | | | |
|---|---|---|---|---|---|
| | concentration | concentration (ppm) | | | Osmolality |
| Name | (mM) | Polysorbate 80 | Pluronic F68 | pH | (mOsmol/kg) |
| F1 | 215 mM | 0 | 0 | 4.9 | 335 |
| F2 | | 10 | 0 | 4.8 | 340 |
| F3 | | 0 | 10 | 4.9 | 342 |
| F4 | | 20 | 0 | 4.8 | 336 |
| F5 | | 0 | 20 | 4.8 | 346 |
| F6 | | 30 | 0 | 4.8 | 338 |
| F7 | | 0 | 30 | 4.8 | 348 |

The compositions are subjected to stirring stress on a magnetic plate at 440 rpm for 6 h, the different analyses carried out thereafter are as follows:
Turbidity (OD at 400 nm): turbidity is determined by measuring the absorbance at 400 nm. Water for injection is used as a blank.

The results obtained are as follows:

TABLE 2
OD measurement at 400 nm before and after
2 h, 4 h and 6 h of stirring stress

| Compositions | T0 | T2 h | T4 h | T6 h |
|---|---|---|---|---|
| F1 | 0.031 | 0.038 | 0.060 | 0.122 |
| F2 | 0.030 | 0.036 | 0.041 | 0.085 |
| F3 | 0.026 | 0.046 | 0.047 | 0.043 |
| F4 | 0.030 | 0.037 | 0.034 | 0.067 |
| F5 | 0.026 | 0.030 | 0.038 | 0.042 |
| F6 | 0.030 | 0.037 | 0.038 | 0.066 |
| F7 | 0.026 | 0.030 | 0.039 | 0.043 |

DLS/SLS: this technique monitors the aggregation state of the solution. Dynamic light scattering (DLS) gives the measurement of the size (hydrodynamic diameter) of objects in solution, approximately between 1 nm and 1 μm. Each size population is thus monitored. Static light scattering (SLS) monitors the aggregation phenomenon as a whole. The measurement is performed by adding 40 mM NaCl to the solution. The measurement is performed on a sample diluted to 80 g/L in water for injection.

The results obtained are as follows:

TABLE 3
Monomer intensity at 90° (%) before
and after 2 h, 4 h and 6 h of stirring stress.

| Formulations | T0 | T2 h | T4 h | T6 h |
|---|---|---|---|---|
| F1 | 97.5 | 92.9 | 77.8 | 65.1 |
| F2 | 98.5 | 93.4 | 90.1 | 54.2 |
| F3 | 92.5 | 75.1 | 76.6 | 68.7 |
| F4 | 98.5 | 90.4 | 90.4 | 67.9 |
| F5 | 95.8 | 73.7 | 82.8 | 84.5 |
| F6 | 96.5 | 92.4 | 88.2 | 67.9 |
| F7 | 94.1 | 83.1 | 81.2 | 65.5 |

TABLE 4
Total intensity diffused at 90° (a.u.) before
and after 2 h, 4 h and 6 h of stirring stress.

| Compositions | T0 | T2 h | T4 h | T6 h |
|---|---|---|---|---|
| F1 | 31.35 | 28.47 | 35.89 | 46.00 |
| F2 | 29.53 | 34.68 | 36.33 | 42.22 |
| F3 | 25.76 | 32.22 | 44.45 | 44.03 |
| F4 | 31.55 | 34.44 | 35.98 | 43.97 |
| F5 | 32.22 | 30.88 | 40.14 | 38.21 |
| F6 | 30.66 | 35.98 | 36.64 | 43.97 |
| F7 | 31.42 | 25.39 | 40.64 | 42.50 |

Subvisible particles (MFI): subvisible particles larger than 2 μm, 10 μm and 25 μm are counted using the microfluidic imaging technique.

The results obtained are as follows:

TABLE 5
Subvisible particles measured by MFI before
and after 2 h, 4 h and 6 h of stirring stress

| | Particles | | | | |
|---|---|---|---|---|---|
| Compositions | (/mL) | T0 | T2 h | T4 h | T8 h |
| F1 | ≥10 μm | 27 | 2404 | 7642 | 39980 |
| | ≥25 μm | 2 | 201 | 362 | 2999 |
| F2 | ≥10 μm | 5 | 1435 | 3263 | 3221 |
| | ≥25 μm | 0 | 94 | 292 | 93 |
| F3 | ≥10 μm | 12 | 339 | 3991 | 3191 |
| | ≥25 μm | 3 | 93 | 233 | 1032 |
| F4 | ≥10 μm | 19 | 949 | 927 | 8043 |
| | ≥25 μm | 1 | 74 | 61 | 1322 |
| F5 | ≥10 μm | 22 | 101 | 936 | 3493 |
| | ≥25 μm | 2 | 17 | 30 | 10 |
| F6 | ≥10 μm | 7 | 477 | 1380 | 15908 |
| | ≥25 μm | 1 | 32 | 52 | 3496 |
| F7 | ≥10 μm | 19 | 134 | 208 | 161 |
| | ≥25 μm | 1 | 10 | 32 | 13 |

The above set of results makes it possible to evaluate the overall stability of the compositions which must show satisfactory results for all the parameters studied.

The results show that compositions F1, F2 and F3, with 0 or 10 ppm surfactant, show unsatisfactory overall stability results, particularly on the measurement of aggregation (DLS) and subvisible particles (MFI), demonstrating a low stability of the composition.

Compositions F6 and F7 comprising 30 ppm surfactant show unsatisfactory results, particularly on the measurement of aggregation (DLS) and subvisible particles (MFI), demonstrating a low stability of the composition.

Conversely, the compositions according to the invention F4 and F5 comprising 20 ppm surfactant make it possible to obtain satisfactory stability on all the criteria evaluated.

Conclusion: the results obtained by different complementary analytical methods demonstrate that a concentration of 20 ppm polysorbate 80 or Pluronic F68® is effective in ensuring the stability of the immunoglobulin composition at 200 g/L in the presence of 215 mM glycine at pH 4.8.

### Example 2: Compositions Placed in Long-Term Stability

An immunoglobulin G composition is prepared according to the process as described in the application EP1385886. The product obtained is then concentrated to 200 g/L by ultrafiltration on Ultracel C cellulose membrane (Millipore®) with a 30 kDa cut-off in order to obtain the ready-to-formulate product (RFP).

Glycine, polysorbate 80 or Pluronic F68 is added to the ready-to-formulate 200 g/L concentrates which are adjusted to the desired pH to obtain the following compositions for stability:

TABLE 6
IgG compositions

| | Glycine | Nonionic detergent | | | |
|---|---|---|---|---|---|
| | concentration | concentration (ppm) | | | Osmolality |
| Name | (mM) | Polysorbate 80 | Pluronic F68 | pH | (mOsmol/kg) |
| F1 | 215 mM | 0 | 0 | 4.9 | 335 |
| F2 | | 10 | 0 | 4.8 | 340 |
| F4 | | 20 | 0 | 4.8 | 336 |
| F5 | | 0 | 20 | 4.8 | 346 |

After sterilizing filtration on a 0.22 μm filter (Sartopore), the compositions are aseptically dispensed into glass vials (type I), which are then capped and stored in a chamber set at
25° C.±2° C./residual humidity 60%±5%, or
40° C.±2° C.

The different analyses carried out are as follows:
Visual inspection: opalescence and visible particle formation are determined by visual inspection using a European Pharmacopoeia test pattern.
pH: change in pH can be a sign of degradation of the product. The pH is measured directly in the solution
Total protein concentration: the absorbance at 280 nm allows according to the Beer-Lambert law the concentration of total proteins in the formulation to be determined. The test is carried out in triplicate, in UV microcells after dilution to 1/400 in water for injection by weighing the samples. The molecular extinction coefficient for the product is 1.4 L/g/cm.
Turbidity (OD at 400 nm): turbidity is determined by measuring absorbance at 400 nm. Water for injection is used as a blank.
DLS/SLS: this technique monitors the aggregation state of the solution. Dynamic light scattering (DLS) gives the measurement of the size (hydrodynamic diameter) of objects in solution, about between 1 nm and 1 μm. Each size population is thus monitored. Static light scattering (SLS) monitors the aggregation phenomenon as a whole. The measurement is performed by adding 40 mM NaCl to the solution. The measurement is performed on a sample diluted to 80 g/L in water for injection.
Subvisible particles (MFI): subvisible particles larger than 2 μm, 10 μm and 25 μm are counted using the microfluidic imaging technique.
HPSEC: high-performance size-exclusion chromatography is used to assess the level of fragmentation and aggregation of the product. Chromatogram analysis of optical density measurements at 208 nm determines the % of monomers, dimers, polymers and fragments.

The results obtained are as follows:

Formulation F1

TABLE 7
Stability results at 25° C. between T0 and T12 months for composition F1

| Stability at +25° C. | Analysis | T0 | T3 months | T6 months | T9 months | T12 months |
|---|---|---|---|---|---|---|
| Visual inspection | Particles - Ph. Eur | Particles | Particles | Particles | Particles | |
| | | free | free | | | |
| | Opalescence - Ph. Eur | Very | Slightly | Very | Slightly | |
| | | slightly | opalescent | slightly | opalescent | |
| | | opalescent | | opalescent | | |
| MFI | total particles/mL | 1950 | 1517 | 34125 | | |
| | particles ≥ 10 μm/mL | 25 | 46 | 2168 | | |
| | particles ≥ 25 μm/mL | 3 | 8 | 123 | | |
| | particles ≥ 10 μm/vial | 382 | 695 | 32514 | | |
| | (vial 15-mL) | | | | | |
| | particles ≥ 25 μm/vial | 52 | 122 | 1841 | | |
| | (vial 15-mL) | | | | | |
| DLS/SLS (% | % monomers - | 94.5 | 90.8 | 89.0 | | |
| monomers) | population at 90° | | | | | |
| | Total intensity at 90° | 32.6 | 32.8 | 31.7 | | |
| | (a.u.) | | | | | |
| turbidity | OD at 400 nm | 0.023 | 0.044 | 0.057 | | |
| Protein | OD at 280 nm | 210.5 | 210.0 | 206.7 | | |
| concentration | | | | | | |
| pH | | 4.8 | 4.8 | 4.8 | | |
| HPSEC | % monomers | | | 98.0 | | |
| | % dimers | | | | | |
| | % polymers | | | 0.6 | | |
| | % fragments | | | 1.5 | | |

TABLE 8
Stability results at 40° C. between T0 and T3 months for composition F1

| Stability at +40° C. | Analysis | T0 | T1 month | T2 months | T3 months |
|---|---|---|---|---|---|
| Visual inspection | Particles - Ph. Eur | Particles | Particles | Particles | Particles |
| | | free | | free | free |
| | Opalescence - Ph. Eur | Very | Very | Slightly | Opalescent |
| | | slightly | slightly | opalescent | |
| | | opalescent | opalescent | | |
| MFI | total particles/mL | 1950 | 29911 | 9131 | 748 |
| | particles ≥ 10 μm/mL | 25 | 4706 | 251 | 68 |
| | particles ≥ 25 μm/mL | 3 | 1836 | 27 | 21 |
| | particles ≥ 10 μm/vial | 382 | 70558 | 3769 | 1025 |
| | (vial 15-mL) | | | | |
| | particles ≥ 25 μm/vial | 52 | 27537 | 399 | 313 |
| | (vial 15-mL) | | | | |
| DLS/SLS (% | % monomers - | 94.5 | 70.3 | 52.9 | 45.5 |
| monomers) | population at 90° | | | | |
| | Total intensity at 90° | 32.6 | 45.6 | 53.5 | 68.2 |
| | (a.u.) | | | | |
| Turbidity | OD at 400 nm | 0.023 | 0.059 | 0.076 | 0.086 |
| Protein | OD at 280 nm | 210.5 | 209.3 | 210.7 | 210.8 |
| concentration | | | | | |
| pH | | 4.8 | 4.9 | 4.9 | 4.9 |

Formulation F2

TABLE 9
Stability results at 25° C. between T0 and T12 months for composition F2

| Stability at +25° C. | Analysis | T0 | T3 months | T6 months | T9 months | T12 months |
|---|---|---|---|---|---|---|
| Visual inspection | Particles - Ph. Eur | Particles | Particles | Few | Particles | Few |
| | | free | free | particles | free | particles |
| | Opalescence - Ph. Eur | Very | Slightly | Very | Very | Very |
| | | slightly | opalescent | slightly | slightly | slightly |
| | | opalescent | | opalescent | opalescent | opalescent |
| MFI | total particles/mL | 129 | 1755 | 1433 | | 2090 |
| | particles ≥ 10 μm/mL | 15 | 84 | 146 | | 137 |
| | particles ≥ 25 μm/mL | 0 | 2 | 13 | | 17 |
| | particles ≥ 10 μm/vial | 226 | 1255 | 2188 | | 2061 |
| | (vial 15-mL) | | | | | |
| | particles ≥ 25 μm/vial | 0 | 24 | 191 | | 258 |
| | (vial 15-mL) | | | | | |
| DLS/SLS (% | % monomers - | 97.3 | 98.3 | 97.7 | | 85.0 |
| monomers) | population at 90° | | | | | |
| | Total intensity at 90° | 30.3 | 33.1 | 32.5 | | 36.8 |
| | (a.u.) | | | | | |
| turbidity | OD at 400 nm | 0.022 | 0.044 | 0.053 | | 0.065 |
| Protein | OD at 280 nm | 204.3 | 208.5 | 206.9 | | 210.7 |
| concentration | | | | | | |
| pH | | 4.8 | 4.8 | 4.8 | | 4.8 |
| HPSEC | % monomers | | | 98.0 | | |
| | % dimers | | | | | |
| | % polymers | | | 0.6 | | |
| | % fragments | | | 1.5 | | |

TABLE 10
Stability results at 40° C. between T0 and T3 months for composition F2

| Stability at +40° C. | Analysis | T0 | T1 month | T2 months | T3 months |
|---|---|---|---|---|---|
| Visual inspection | Particles - Ph. Eur | Particles | Particles | Particles | Particles |
| | | free | free | free | free |
| | Opalescence - Ph. Eur | Very | Very | Slightly | Opalescent |
| | | slightly | slightly | opalescent | |
| | | opalescent | opalescent | | |
| MFI | total particles/mL | 129 | 460 | 2653 | 1271 |
| | particles ≥ 10 μm/mL | 15 | 19 | 29 | 46 |
| | particles ≥ 25 μm/mL | 0 | 17 | 1 | 0 |
| | particles ≥ 10 μm/vial | 226 | 278 | 434 | 695 |
| | (vial 15-mL) | | | | |
| | particles ≥ 25 μm/vial | 0 | 17 | 17 | 0 |
| | (vial 15-mL) | | | | |
| DLS/SLS (% | % monomers - | 97.3 | 70.2 | 53.2 | 42.9 |
| monomers) | population at 90° | | | | |
| | Total intensity at 90° | 30.3 | 44.4 | 56.3 | 74.4 |
| | (a.u.) | | | | |
| Turbidity | OD at 400 nm | 0.022 | 0.055 | 0.075 | 0.087 |
| Protein | OD at 280 nm | 204.3 | 207.9 | 211.6 | 206.7 |
| concentration | | | | | |
| pH | | 4.8 | 4.9 | 4.9 | 4.9 |

Formulation F4

TABLE 11
Stability results at 25° C. at T0 between T12 months for composition F4

| Stability at +25° C. | Analysis | T0 | T3 months | T6 months | T9 months | T12 months |
|---|---|---|---|---|---|---|
| Visual inspection | Particles - Ph. Eur | Particles | Particles | Particles | Particles | Particles |
| | | free | free | free | free | free |
| | Opalescence - Ph. Eur | Very | Slightly | Very | Very | Very |
| | | slightly | opalescent | slightly | slightly | slightly |
| | | opalescent | | opalescent | opalescent | opalescent |
| MFI | total particles/mL | 316 | 932 | 1040 | | 1724 |
| | particles ≥ 10 μm/mL | 9 | 34 | 37 | | 59 |
| | particles ≥ 25 μm/mL | 3 | 3 | 12 | | 1 |
| | particles ≥ 10 μm/vial | 139 | 504 | 556 | | 886 |
| | (vial 15-mL) | | | | | |
| | particles ≥ 25 μm/vial | 52 | 52 | 174 | | 17 |
| | (vial 15-mL) | | | | | |
| DLS/SLS (% | % monomers - | 97.2 | 94.8 | 95.6 | | 82.6 |
| monomers) | population at 90° | | | | | |
| | Total intensity at 90° | 30.5 | 34.2 | 34.0 | | 37.1 |
| | (a.u.) | | | | | |
| turbidity | OD at 400 nm | 0.022 | 0.043 | 0.052 | | 0.066 |
| Protein | OD at 280 nm | 204.4 | 207.6 | 207.3 | | 209.0 |
| concentration | | | | | | |
| pH | | 4.8 | 4.8 | 4.8 | | 4.8 |
| HPSEC | % monomers | | | 98.0 | | |
| | % dimers | | | | | |
| | % polymers | | | 0.6 | | |
| | % fragments | | | 1.5 | | |

TABLE 12
Stability results at 40° C. between T0 and T3 months for composition F4

| Stability at +40° C. | Analysis | T0 | T1 month | T2 months | T3 months |
|---|---|---|---|---|---|
| Visual inspection | Particles - Ph. Eur | Particles | Particles | Particles | Particles |
| | | free | free | free | free |
| | Opalescence - Ph. Eur | Very | Very | Slightly | Opalescent |
| | | slightly | slightly | opalescent | |
| | | opalescent | opalescent | | |
| MFI | total particles/mL | 316 | 442 | 1527 | 1198 |
| | particles ≥ 10 μm/mL | 9 | 69 | 25 | 10 |
| | particles ≥ 25 μm/mL | 3 | 28 | 0 | 1 |
| | particles ≥ 10 μm/vial | 139 | 1042 | 382 | 156 |
| | (vial 15-mL) | | | | |
| | particles ≥ 25 μm/vial | 52 | 417 | 0 | 17 |
| | (vial 15-mL) | | | | |
| DLS/SLS (% | % monomers - | 97.2 | 66.4 | 52.1 | 44.2 |
| monomers) | population at 90° | | | | |
| | Total intensity at 90° | 30.5 | 42.1 | 57.1 | 75.2 |
| | (a.u.) | | | | |
| Turbidity | OD at 400 nm | 0.022 | 0.057 | 0.076 | 0.088 |
| Protein | OD at 280 nm | 204.4 | 207.5 | 210.8 | 209.0 |
| concentration | | | | | |
| pH | | 4.8 | 4.9 | 4.9 | 4.9 |

Formulation F5

TABLE 13
Stability results at 25° C. between T0 and T12 months for composition F5

| Stability at +25° C. | Analysis | T0 | T3 months | T6 months | T9 months | T12 months |
|---|---|---|---|---|---|---|
| Visual inspection | Particles - Ph. Eur | Particles | Particles | Particles | Particles | Particles |
| | | free | free | free | free | free |
| | Opalescence - Ph. Eur | Very | Very | Very | Very | Very |
| | | slightly | slightly | slightly | slightly | slightly |
| | | opalescent | opalescent | opalescent | opalescent | opalescent |
| MFI | total particles/mL | 1300 | 506 | 729 | | 1057 |
| | particles ≥ 10 μm/mL | 20 | 15 | 45 | | 98 |
| | particles ≥ 25 μm/mL | 2 | 3 | 7 | | 21 |
| | particles ≥ 10 μm/vial | 295 | 226 | 677 | | 1476 |
| | (vial 15-mL) | | | | | |
| | particles ≥ 25 μm/vial | 35 | 52 | 104 | | 313 |
| | (vial 15-mL) | | | | | |
| DLS/SLS (% | % monomers - | 96.8 | 98.6 | 96.2 | | 90.4 |
| monomers) | population at 90° | | | | | |
| | Total intensity at 90° | 32.4 | 31.0 | 31.5 | | 35.0 |
| | (a.u.) | | | | | |
| turbidity | OD at 400 nm | 0.028 | 0.046 | 0.053 | | 0.067 |
| Protein | OD at 280 nm | 210.3 | 208.6 | 209.9 | | 212.5 |
| concentration | | | | | | |
| pH | | 4.8 | 4.9 | 4.9 | | 4.9 |
| HPSEC | % monomers | | | 98.1 | | |
| | % dimers | | | | | |
| | % polymers | | | 0.5 | | |
| | % fragments | | | 1.4 | | |

TABLE 14
Stability results at 40° C. between T0 and T3 months for composition F5

| Stability at +40° C. | Analysis | T0 | T1 month | T2 months | T3 months |
|---|---|---|---|---|---|
| Visual inspection | Particles - Ph. Eur | Particles | Particles | Particles | Particles |
| | | free | | free | free |
| | Opalescence - Ph. Eur | Very | Very | Opalescent | Opalescent |
| | | slightly | slightly | Slight | Slight |
| | | opalescent | opalescent | | |
| MFI | total particles/mL | 1300 | 1333 | 2304 | 867 |
| | particles ≥ 10 μm/mL | 20 | 60 | 1035 | 36 |
| | particles ≥ 25 μm/mL | 2 | 14 | 1 | 6 |
| | particles ≥ 10 μm/vial | 295 | 903 | 15527 | 538 |
| | (vial 15-mL) | | | | |
| | particles ≥ 25 μm/vial | 35 | 208 | 17 | 87 |
| | (vial 15-mL) | | | | |
| DLS/SLS (% | % monomers - | 96.8 | 73.5 | 56.9 | 47.2 |
| monomers) | population at 90° | | | | |
| | Total intensity at 90° | 32.4 | 45.0 | 54.5 | 71.2 |
| | (a.u.) | | | | |
| Turbidity | OD at 400 nm | 0.028 | 0.054 | 0.072 | 0.091 |
| Protein | OD at 280 nm | 210.3 | 209.4 | 211.8 | 210.7 |
| concentration | | | | | |
| pH | | 4.8 | 4.9 | 5.0 | 4.9 |

Tests at 40° C. accelerate the phenomena observed during the stability of the compositions due to the high stress applied. The methods used clearly show a change in the criteria monitored, confirming the relevance of the methods selected for assessing formulations during stability monitoring.

Long-term stability results show that formulations F1 and F2 with 0 or 10 ppm surfactant do not maintain a particle-free composition above 25° C., demonstrating that the stability of these compositions is unsatisfactory.

On the other hand, the formulations according to the invention F4 and F5 with 20 ppm surfactant (Polysorbate 80 or Pluronic F68) maintain the compositions stable over time.

1.-14. (canceled)

15. A pharmaceutical composition, comprising:
200 g/L, ±5% immunoglobulin G (IgG),
between 200 and 250 mM glycine, and
between 15 and 25 ppm nonionic detergent,
wherein the composition has pH between 4.6 and 5.0.

16. The pharmaceutical composition of claim 15, wherein the IgG are human IgG.

17. The pharmaceutical composition of claim 15, wherein glycine is present at a concentration between 200 and 230 mM.

18. The pharmaceutical composition of claim 17, wherein glycine is present at a concentration of 215 mM, ±5%.

19. The pharmaceutical composition of claim 15, wherein the nonionic detergent is present at a concentration between 15 and 25 ppm.

20. The pharmaceutical composition of claim 19, wherein the nonionic detergent is present at a concentration of 20 ppm, ±10%.

21. The pharmaceutical composition of claim 15, wherein the nonionic detergent is selected from the group consisting of polysorbates and poloxamers.

22. The pharmaceutical composition of claim 21, wherein the nonionic detergent is polysorbate 20 or polysorbate 80.

23. The pharmaceutical composition of claim 15, wherein the nonionic detergent is Pluronic F68.

24. The pharmaceutical composition of claim 15, wherein the pH is 4.8±0.1.

25. The pharmaceutical composition of claim 15, wherein the composition is devoid of acetate salts and/or mannitol and/or albumin.

26. The pharmaceutical composition of claim 15, comprising:
200 g/L, ±5% IgG,
215 mM, ±5% glycine, and
20 ppm, ±10% nonionic detergent,
wherein the composition has pH 4.8±0.1.

27. The pharmaceutical composition of claim 15, wherein the composition is in liquid form.

28. The pharmaceutical composition of claim 15, wherein the composition has an osmolality between 300 and 400 mOsm/kg.

29. The pharmaceutical composition of claim 28, wherein the osmolality is about 240 mOsm/kg.

30. The pharmaceutical composition of claim 15, wherein the IgG is obtained by fractionation of blood plasma.
